(19) United States
(12) Patent Application Publication
(10) Pub. No.: US 2008/0064086 A1
(43) Pub. Date: Mar. 13, 2008

(54) PLASTIC-BASED MICROFABRICATED THERMAL DEVICE, MANUFACTURING METHOD THEREOF, DNA AMPLIFICATION CHIP USING THE PLASTIC-BASED MICROFABRICATED THERMAL DEVICE, AND METHOD FOR MANUFACTURING THE DNA AMPLIFICATION CHIP

(75) Inventors: Dae-Sik Lee, Daejon (KR); Se-Ho Park, Daejon (KR); Kwang-Hyo Chung, Daejon (KR); Hyeon-Bong Pyo, Daejon (KR)

Correspondence Address: RABIN & Berdo, PC, 1101 14TH STREET, NW, SUITE 500, WASHINGTON, DC, 20005, US

(73) Assignee: Electronics and Telecommunications Research Institute, Daejon (KR)

(21) Appl. No.: 11/854,401

(22) Filed: Sep. 12, 2007

(30) Foreign Application Priority Data
Sep. 13, 2006 (KR) 10-2006-0088453

(51) Int. Cl. C12M 1/02; B05D 3/00; H05B 3/03; B05D 5/00

(52) U.S. Cl. 435/289.1; 219/520; 427/123; 427/58; 432/227

(57) ABSTRACT

Provided are a microfabricated thermal device using a thin plastic substrate, a manufacturing method thereof, a silicon micro-chamber, a double-stranded deoxyribonucleic acid (DNA) amplification chip employing the microfabricated thermal device and the silicon micro-chamber, and a manufacturing method thereof, and a DNA amplification chip array, and a method for manufacturing the DNA amplification chip array. The microfabricated thermal device using a thin plastic substrate can be used for DNA amplification, i.e., a polymerase chain reaction (PCR), which is essential to DNA related diagnosis and analysis. The plastic-based microfabricated thermal device, includes: a plastic substrate; a heating unit disposed on the top surface of the plastic substrate to supply heat to the plastic substrate; a sensing unit disposed on the top surface of the plastic substrate to detect heat; and a diffusing unit disposed on the bottom surface of the plastic substrate to diffuse heat to the plastic substrate.

### CROSS-REFERENCE(S) TO RELATED APPLICATIONS

The present invention claims priority of Korean Patent Application No. 10-2006-0088453, filed on Sep. 13, 2006 which is incorporated herein by reference.

### BACKGROUND OF THE INVENTION

1. Field of the Invention

The present invention relates to a bio-micro electric-mechanical system (Bio-MEMS); and, more particularly, to a microfabricated thermal device using a thin plastic substrate, which can be used for double-stranded deoxyribonucleic acid (DNA) amplification, i.e., a polymerase chain reaction (PCR), which is essential to DNA related diagnosis and analysis, and a manufacturing method thereof, a silicon micro-chamber and a manufacturing method thereof, a DNA amplification chip and a manufacturing method thereof, and a DNA amplification chip array and a manufacturing method thereof.

2. Description of Related Art

With rapid development of Biotechnologies, studies have been intensively conducted on medical micro-devices, called a lab-on-a-chip, which use DNA to diagnose a variety of diseases. Many efforts have been directed to miniaturization and low cost of medical micro-devices in order for real-time diagnosis and disposable use.

A DNA micro-device among medical micro-devices requires heating of DNA at high temperature. Specifically, it is necessary to heat DNA at a temperature ranging from approximately 40° C. to approximately 100° C. for the purposes of cell decomposition, DNA amplification, i.e., PCR, reaction regulation, fluid delivery, etc. A variety of microfabricated thermal devices for processing DNA have been developed. In most cases, silicon and glass are used.

Since The DNA micro-devices should have low power consumption, it is suitable for portable battery, and short analysis time for real-time diagnosis. To this end, thermal isolation should be possible and a structure having small thermal mass should be designed and manufactured. Such a structure has been manufactured using silicon-based semiconductor fabrication technology. The reason for this is that the semiconductor fabrication technology is well established and can form fine patterns.

Thermocyclers having a plurality of chambers are disclosed in U.S. Pat. No. 5,589,136, issued to M. Allen Northrup et al on Dec. 31, 1996, U.S. Pat. No. 6,503,750, issued to William J. Benett et al on Feb. 10, 1998, and Korean Patent No. 10-0450818, issued to Yoon D et al on Sep. 20, 2004. In these patents, a DNA amplification chip is manufactured by photolithography and silicon etching processes of forming a heating wire and a temperature sensor on a semiconductor substrate.

Although heaters can be implemented in reaction chambers by using these technologies, it is difficult to eliminate thermal crosstalk because of limited thermal isolation between reaction chambers. Hence, these technologies are difficult to apply to chambers having independent temperature cycles. In addition, although the use of silicon can obtain the excellent performance of devices, the semiconductor fabrication technology requires very clean laboratories and very expensive apparatuses, so that a manufacturing cost increases and a manufacturing process takes a long time. Hence, these technologies are difficult to apply to disposable diagnosis instruments.

Another technology is disclosed in “Analytic Chemistry” Journal, R.A. Mathies group of Berkeley University of California, Feb. 1, 2001, entitled “single-molecule DNA amplification and analysis in an integrated microfluidic device”. In this journal, a system having a capillary electrophoresis (CE) and a reaction chamber is manufactured using a glass substrate, and a PCR is carried out on the glass substrate. However, since this technology has difficulty in processing the glass substrate, it cannot form a heating thin film having small thermal mass. Therefore, a proportion-integration-derivation (PID) controller should be separately provided because of high power consumption and slow reaction speed.

As described above, the use of silicon has disadvantages in that the thermal isolation characteristic is poor and the processing of the substrate is difficult. Further, a manufacturing cost increases because silicon or glass is expensive. Therefore, there is a need for materials that have thermal characteristic comparable to silicon or glass, are cheaper than silicon or glass, and are easy to process.

### SUMMARY OF THE INVENTION

An embodiment of the present invention is directed to providing a microfabricated thermal device, which is cheaper than silicon or glass to thereby reduce a manufacturing cost and can achieve a uniform temperature control, and a method for manufacturing the same.

Another embodiment of the present invention is directed to provide a microfabricated thermal device, which can reduce a thermal mass, and a method for manufacturing the same.

Another embodiment of the present invention is directed to providing a microfabricated thermal device, can be manufactured using well-known semiconductor fabrication technologies, and a method for manufacturing the same.

Another embodiment of the present invention is directed to providing a microfabricated thermal device, which can increase thermal uniformity, and a method for manufacturing the same.

Another embodiment of the present invention is directed to providing a silicon micro-chamber having a reaction chamber, which is capable of enhancing thermal uniformity and response time, and a method for manufacturing the same.

Another embodiment of the present invention is directed to provide a DNA amplification chip, which is manufactured by combining the microfabricated thermal device and the silicon micro-chamber, and a method for manufacturing the same.

Another embodiment of the present invention is to provide a DNA amplification chip array having a plurality of DNA amplification chips, and a method for manufacturing the same.

In accordance with an aspect of the present invention, there is provided a plastic-based microfabricated thermal device, which includes: a plastic substrate; a heating unit disposed on the top surface of the plastic substrate to supply heat to the plastic substrate; a sensing unit disposed on the top surface of the plastic substrate to detect heat; and a diffusing unit disposed on the bottom surface of the plastic substrate to diffuse heat to the plastic substrate.

In accordance with another aspect of the present invention, there is provided a DNA amplification chip, which includes: a plastic-based microfabricated thermal device including a plastic substrate, a heating unit disposed on the top surface of the plastic substrate to supply heat to the plastic substrate, a sensing unit disposed on the top surface of the plastic substrate to detect heat, and a diffusing unit disposed on the bottom surface of the plastic substrate to diffuse heat to the plastic substrate; a silicon micro-chamber including a concave region and attached to the microfabricated thermal device, with the concave region being directed upwards; and a cover disposed to cover the concave region of the silicon micro-chamber, thereby defining a reaction chamber.

In accordance with another aspect of the present invention, there is provided a method for manufacturing a plastic-based microfabricated thermal device, which includes the steps of: a) preparing a plastic substrate; b) forming a heater, an electrode, a pad, and a temperature sensor on the top surface of the plastic substrate; c) forming a heat diffusion layer on the bottom surface of the plastic substrate; d) forming insulating layers on the top and bottom surfaces of the plastic substrate to cover the heater, the electrode, the pad, the temperature sensor, and the heat diffusion layer; and e) etching the insulating layers to expose predetermined portions of the electrode and the pad.

In accordance with another aspect of the present invention, there is provided a method for manufacturing a DNA amplification chip, which includes the steps of: a) providing a plastic-based microfabricated thermal device, the plastic-based microfabricated thermal device being formed by preparing a plastic substrate, forming a heater, an electrode, a pad, and a temperature sensor on the top surface of the plastic substrate, forming a heat diffusion layer on the bottom surface of the plastic substrate, forming insulating layers on the top and bottom surfaces of the plastic substrate to cover the heater, the electrode, the pad, the temperature sensor, and the heat diffusion layer, and etching the insulating layers to expose predetermined portions of the electrode and the pad; b) forming a silicon micro-chamber having a concave region and attaching the silicon micro-chamber to the top surface of the microfabricated thermal device; and c) covering the concave region by a cover to form a reaction chamber.

Other objects and advantages of the present invention can be understood by the following description, and become apparent with reference to the embodiments of the present invention. Also, it is obvious to those skilled in the art to which the present invention pertains that the objects and advantages of the present invention can be realized by the means as claimed and combinations thereof.

### BRIEF DESCRIPTION OF THE DRAWINGS

FIG. 1 is a cross-sectional view illustrating a microfabricated thermal device in accordance with a first embodiment of the present invention.

FIGS. 2A to 2E are cross-sectional views illustrating a method for manufacturing the microfabricated thermal device illustrating in FIG. 1.

FIG. 3 is a cross-sectional view illustrating a silicon micro-chamber in accordance with a second embodiment of the present invention.

FIGS. 4A to 4C are cross-sectional views illustrating a method for manufacturing the silicon micro-chamber illustrated in FIG. 3.

FIG. 5 is a cross-sectional view illustrating a double-stranded DNA amplification chip in accordance with a third embodiment of the present invention.

FIG. 6 is a cross-sectional view illustrating a DNA amplification chip array having a plurality of DNA amplification chips illustrated in FIG. 5.

FIG. 7 is a cross-sectional view illustrating a DNA amplification chip in accordance with a fourth embodiment of the present invention.

FIG. 8 is a cross-sectional view illustrating a DNA amplification array having a plurality of DNA amplification chips illustrated in FIG. 7.

FIG. 9A is a photograph of the microfabricated thermal device illustrated in FIG. 1.

FIG. 9B is a photograph of the DNA amplification chip illustrated in FIG. 5.

FIG. 10 is a graph illustrating a temperature-time response characteristic of a typical PCR method.

FIG. 11 is a photograph illustrating comparative analysis of PCR results, which are obtained using a fluorescent photography through an electrophoresis, before and after a temperature control of PCR is performed on the DNA amplification chip of FIG. 5, and after the temperature control is performed in a mechanical PCR apparatus.

### DESCRIPTION OF SPECIFIC EMBODIMENTS

The advantages, features and aspects of the invention will become apparent from the following description of the embodiments with reference to the accompanying drawings, which is set forth hereinafter.

### Embodiment 1

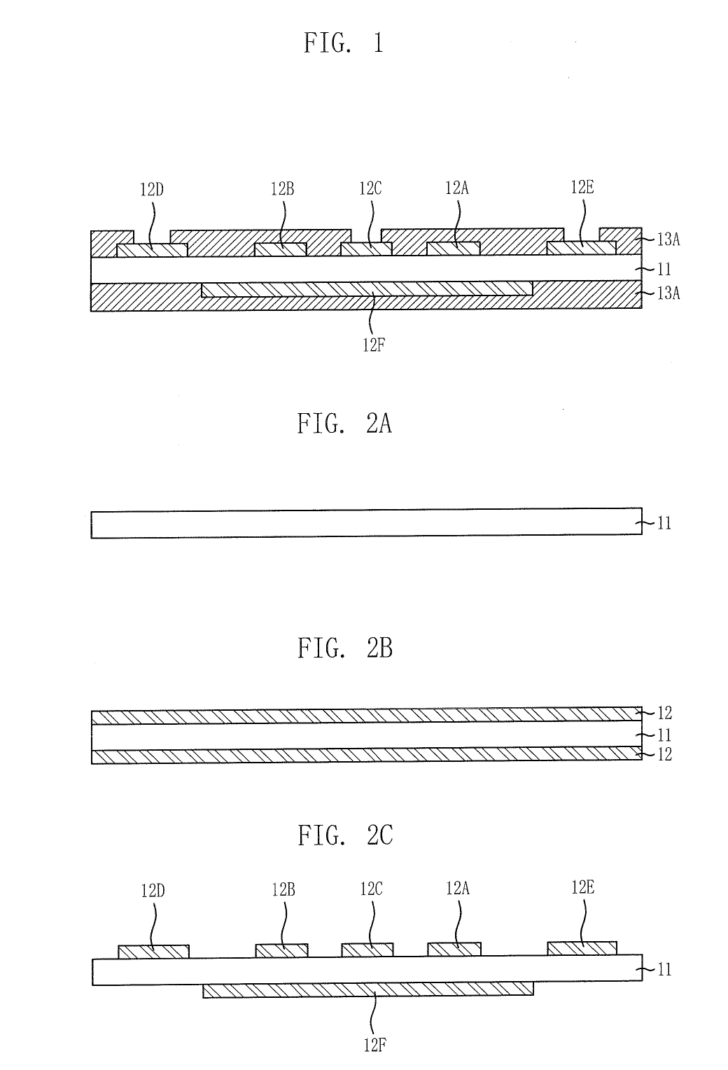

FIG. 1 is a cross-sectional view illustrating a microfabricated thermal device in accordance with a first embodiment of the present invention.

Referring to FIG. 1, the microfabricated thermal device includes a heater 12A, a temperature sensor 12B, an electrode 12C, and a plurality of pads 12D and 12E, which are sequentially formed on a plastic substrate 11. In addition, the microfabricated thermal device further includes a heat diffusion layer 13A on the bottom surface of the plastic substrate 11. Insulating layers 13A are formed on the top and bottom surfaces of the plastic substrate 11 to cover the heater 12A, the temperature sensor 12B, the electrode 12C, the pads 12D and 12E, and the heat diffusion layer 12F. The insulating layers 13A are patterned to expose predetermined portions of the electrode 12C and the pads 12D and 12E.

The plastic substrate 11 is formed of plastic, which has a surface roughness of, e.g., 0.1-500 nm at which a photolithography process is applicable, a compatibility with chemicals used in the photolithography process, a small thickness of, e.g., 1-500 μm, a low thermal conductivity, and a small thermal mass. The surface roughness and thickness of the plastic substrate 11 are determined such that fine patterns having a thickness of 0.01 to 5 μm and a line width of 1 to 100 μm can be formed in a wafer. In order for the compatibility with chemicals used in the photolithography process, the plastic substrate 11 may be coated with liquid water glass or organic thin film, e.g., heat-resistant and chemical-resistant organic materials such as epoxy, and then thermally treated.

The plastic substrate 11 may be formed of a polymer such as Cyclo Olefin Copolymer (COC), PolyMethylMethAcrylate (PMMA), PolyCarbonate (PC), Cyclo Olefin Polymer (COP), Liquid Crystalline Polymers (LCP), PolyDiMethylSiloxane (PDMS), PolyAmide (PA), PolyEthylene (PE), PolyImide (PI), PolyPropylene (PP), PolyPhenylene Ether (PPE), PolyStyrene (PS), PolyOxyMethylene (POM), PolyEtherEtherKetone (PEEK), PolyEthylenephThalate (PET), PolyTetraFluoroEthylene (PTFE), PolyVinylChloride (PVC), PolyVinyliDeneFluoride (PVDF), PolyButyleneTerephtalate (PBT), Fluorinated EthyleneproPylene (FEP), and PerFluorAlkoxyalkane (PFA), and mixtures thereof.

The plastic substrate 11 may be formed by an injection molding using a mold processed by a chemical mechanical polishing (CMP), an extrusion molding, a hot embossing or a casting, a stereolithography, a laser ablation, a rapid prototyping, a founding, a silk screen, a machining such as a numerical control machining, or a semiconductor fabrication process such as a photography process and an etching process.

The heater 12A, the temperature sensor 12B, the electrode 12C, the pads 12D and 12E, and the heat diffusion layer 12F may be simultaneously formed of noble metal such as platinum or gold.

The heater 12A supplies heat to the plastic substrate 11, and the electrode 12C and the pads 12D and 12E supply power to the heater 12A. The temperature sensor 12B detects the temperature of the plastic substrate 11.

The heat diffusion layer 12F is formed on the bottom surface of the plastic substrate 11 and uniformly diffuses heat generated from the plastic substrate 11, thereby increasing the overall thermal uniformity of the plastic substrate 11. The heat diffusion layer 12F is formed of a thermally conductive material, such as metal or graphite.

The insulating layers 13A may be formed of an organic or inorganic material. In the case of using the inorganic material, the insulating layers 13A having a thickness of a few to a few tens of μm can be formed of water glass by a spin, spray, or laminating coating process. A rough surface of the plastic substrate 11 can be planarized by a thermal treatment at a temperature of approximately 50 to 300° C. In the case of using the organic material, the insulating layers 13A having a thickness of a few of μm can be formed of an epoxy resin by a spin or spray coating process. A chemical tolerance and heat tolerance of the plastic substrate 11 can be increased by performing a thermal treatment on the insulating layers 13A at a temperature of approximately 50 to 300° C.

Meanwhile, the insulating layer 13A formed on the bottom surface of the plastic substrate 11 serves to insulate the plastic substrate 11 and guide the heat diffused by the heat diffusion layer 12F upward to the plastic substrate 11.

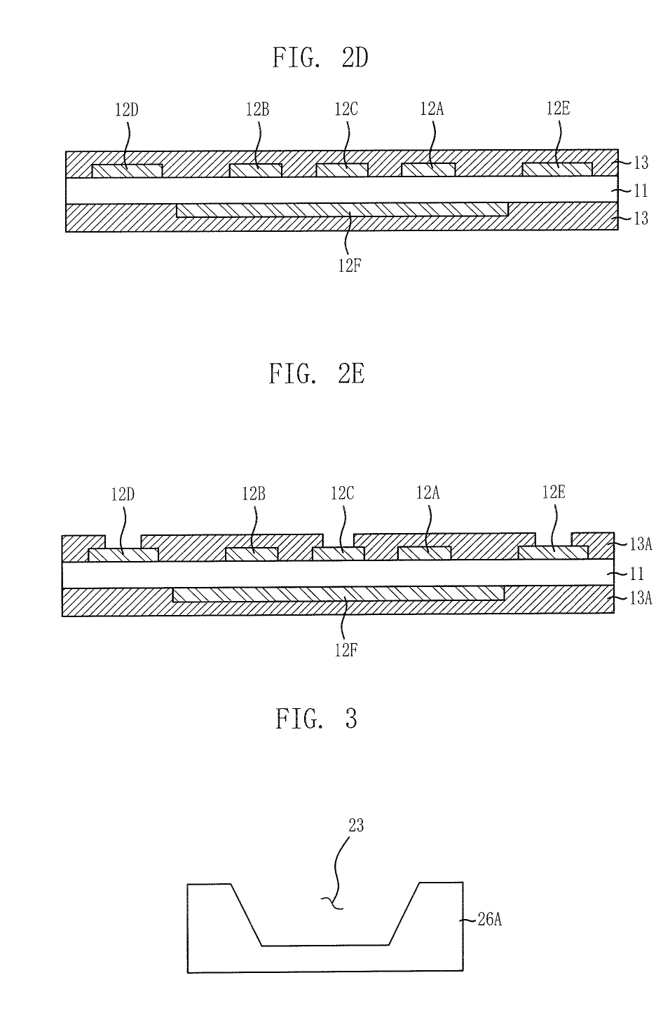

A method for manufacturing the microfabricated thermal device illustrated in FIG. 1 will be described below with reference to FIGS. 2A to 2E.

FIGS. 2A to 2E are cross-sectional views illustrating a method for manufacturing the microfabricated thermal device.

Referring to FIG. 2A, a thin plastic substrate 11 having a thickness of 1 to 500 μm is prepared. The plastic substrate 11 may be formed of a polymer such as Cyclo Olefin Copolymer (COC), PolyMethylMethAcrylate (PMMA), PolyCarbonate (PC), Cyclo Olefin Polymer (COP), Liquid Crystalline Polymers (LCP), PolyDiMethylSiloxane (PDMS), PolyAmide (PA), PolyEthylene (PE), PolyImide (PI), PolyPropylene (PP), PolyPhenylene Ether (PPE), PolyStyrene (PS), PolyOxyMethylene (POM), PolyEtherEtherKetone (PEEK), PolyEthylenephThalate (PET), PolyTetraFluoroEthylene (PTFE), PolyVinylChloride (PVC), PolyVinyliDeneFluoride (PVDF), PolyButyleneTerephtalate (PBT), Fluorinated EthyleneproPylene (FEP), and PerFluorAlkoxyalkane (PFA), and mixtures thereof. Further, the plastic substrate 11 may be formed by an injection molding using a mold processed by a chemical mechanical polishing (CMP), an extrusion molding, a hot embossing or a casting, a stereolithography, a laser ablation, a rapid prototyping, a founding, a silk screen, a machining such as a numerical control machining, or a semiconductor fabrication process such as a photography process and an etching process. In the plastic substrate 11, a heating zone, i.e., a region where a heater (12A in FIG. 2D) and a temperature sensor (12B in FIG. 2D) will be formed, may be partially etched to form a concave region (not shown) in the heating zone. A thermal isolation can be enhanced by forming the heater 12A and the temperature sensor 12B in the concave region.

Since the plastic substrate 11 is very flexible, the semiconductor fabrication process is difficult to carry out. Therefore, the plastic substrate 11 may be fixed to a solid substrate such as silicon or glass wafer by using an adhesive material, which is easy to adhere to or detach from the solid substrate.

Referring to FIG. 2B, metal layers 12 are deposited on the top and bottom surfaces of the plastic substrate 11. The metal layers 12 may be formed of conductive materials. Preferably, the metal layers are formed of noble metals having high thermal conductivity, such as platinum or gold.

Referring to FIG. 2C, the metal layer 12 formed on the top surface of the plastic substrate 11 is etched by a photolithography process and an etching process, thereby forming a heater 12A, a temperature sensor 12B, an electrode 12C, and pads 12D and 12E on the plastic substrate 11.

After the plastic substrate 11 is turned up and down, the metal layer 12 formed on the bottom surface of the plastic substrate 11 is etched by a photolithography process and an etching process, thereby forming a heat diffusion layer 12F on the bottom surface of the plastic substrate 11.

Referring to FIG. 2D, insulating layers 13 are formed on the top and bottom surfaces of the plastic substrate 11 to cover the heater 12A, the temperature sensor 12B, the electrode 12C, the pads 12D and 12E, and the heat diffusion layer 12F. The insulating layers 13 may be formed of an organic or inorganic material. In the case of using the inorganic material, the insulating layers 13 having a thickness of a few to a few tens of μm can be formed of water glass by a spin, spray, or laminating coating process. A rough surface of the plastic substrate 11 can be planarized by a thermal treatment at a temperature of approximately 50 to 300° C. In the case of using the organic material, the insulating layers 13 having a thickness of a few of μm can be formed of an epoxy resin by a spin or spray coating process. A chemical tolerance and heat tolerance of the plastic substrate 11 can be increased by performing a thermal treatment on the insulating layers 13A at a temperature of approximately 50 to 300° C.

Referring to FIG. 2E, a photolithography process and an etching process are sequentially performed to etch the insulating layers (13 in FIG. 2D). Consequently, an insulating layer pattern 13A is formed to expose predetermined portions of the electrode 12C and the pads 12D and 12E, which are formed on the plastic substrate 11. A wet etching process and/or a dry etching process can be used.

The photolithography process is a process of depositing a photoresist layer and forming a photoresist pattern by an exposure process and a development process using a photo mask.

### Embodiment 2

FIG. 3 is a cross-sectional view illustrating a silicon micro-chamber in accordance with a second embodiment of the present invention.

Referring to FIG. 3, the silicon micro-chamber includes a silicon substrate 21A. A thermal uniformity and a response time of the silicon substrate 21A match with those of the plastic-based microfabricated thermal device in accordance with the first embodiment of the present invention. An inlet and an outlet (not shown), a reaction chamber 23, a valve and a mixer (not shown), a passage (not shown) are formed in the silicon substrate 21A. Specifically, fluid is introduced through the inlet and discharged through the outlet in order to control temperature and biological/chemical reaction with respect to microfluid. The fluid reacts within the reaction chamber 23, and the passage connects the inlet and the outlet.

Meanwhile, the reaction chamber 23 is formed in a concave shape at the center of the silicon substrate 21A corresponding to a heating zone of a microfabricated thermal device. Since the concave region is thin compared to other regions, it is thermally isolated and its thermal mass is low. Therefore, a good thermal response characteristic can be obtained.

A method for manufacturing the silicon micro-chamber in accordance with the second embodiment of the present invention will be described below.

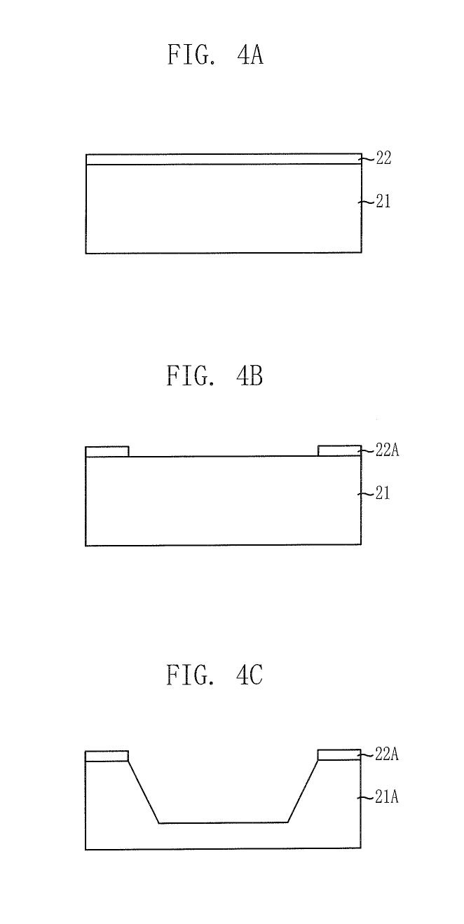

FIGS. 4A to 4C are cross-sectional views illustrating a method for manufacturing the silicon micro-chamber.

Referring to FIG. 4A, an insulating layer 22 is deposited on a silicon substrate 2. The insulating layer 22 is formed of silicon-based oxide, e.g., SiO₂, or silicon-based nitride, e.g., SiON, or a photoresist. For convenience, the insulating layer 22 formed of a photoresist will be described for illustrative purpose.

Referring to FIG. 4B, an exposure process and a development process are sequentially performed using the photoresist layer 22 as a photo mask to form a photoresist pattern 22A.

Referring to FIG. 4C, the silicon substrate (21 in FIG. 4B) is etched by an etching process using the photoresist pattern 22A. A reaction chamber 23 is formed at the center portion corresponding to the heating zone of the microfabricated thermal device 10, i.e., the region where the heater 12A, the temperature sensor 12B, and the electrode 12C are formed. A wet etching process or a dry etching process can be used for forming the reaction chamber 23. In using the wet etching process, potassium hydroxide (KOH) or Tetra-Methyl Ammonium Hydroxide (TMAH) may be used. In using the dry etching process, a deep reactive ion etching (DRIE) process using chemicals such as SF₆ may be carried out.

Although the structure in which the silicon micro-chamber is integrally formed has been described, a support wall surrounding the reaction chamber 23 can be separately formed.

A silicon substrate 21A in which the reaction chamber 23 is formed is illustrated in FIG. 4C.

### Embodiment 3

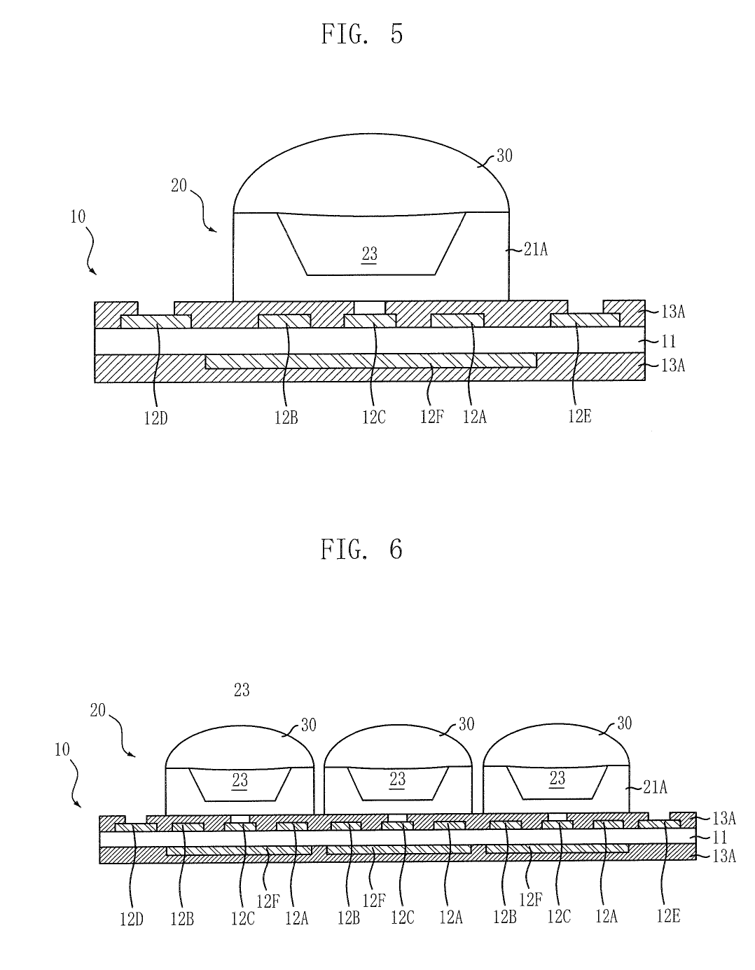

FIG. 5 is a cross-sectional view illustrating a DNA amplification chip and a method for manufacturing the same in accordance with a third embodiment of the present invention.

Referring to FIG. 5, the DNA amplification chip is manufactured by attaching the microfabricated thermal device 10 of FIG. 1 and the silicon micro-chamber 20 of FIG. 3. In addition, a cover 30 formed of inorganic oil is formed over the silicon micro-chamber 20.

A method for manufacturing the DNA amplification chip in accordance with the third embodiment of the present invention will be described below.

Referring to FIG. 5, a silicon micro-chamber 20 is physically attached to the microfabricated thermal device 10. A high conductive material such as a paste or a compound may be used for adhesion and heat conduction. The microfabricated thermal device 10 and the silicon micro-chamber 20 may be forcibly coupled using an additional clip-type structure. Alternatively, a convex protrusion is formed in one of the microfabricated thermal device 10 and the silicon micro-chamber 20, and a concave groove is formed in the other of the microfabricated thermal device 10 and the silicon micro-chamber 20. Then, the microfabricated thermal device 10 and the silicon micro-chamber 20 are coupled to each other by fitting the convex protrusion into the concave groove. In this case, an elastic polymer layer may be further provided in the contact surface between the microfabricated thermal device 10 and the silicon micro-chamber 20 in order to prevent the formation of fine gap.

In order to prevent evaporation of genetic sample during the DAM amplification process using PCR, an inorganic oil cover 30 is coupled to cover the reaction chamber 23 of the silicon micro-chamber 20 attached to the microfabricated thermal device 10. In this way, bubbles generated during the heating within the silicon micro-chamber 20 are discharged and the evaporation of genetic sample is prevented.

FIG. 6 is a cross-sectional view illustrating a DNA amplification chip array in which a plurality of DNA amplification chips of FIG. 5 are arranged. As illustrated in FIG. 6, the DNA amplification chip array can be manufactured in a batch manner by using the method for manufacturing the single DNA amplification chip illustrated in FIG. 5.

### Embodiment 4

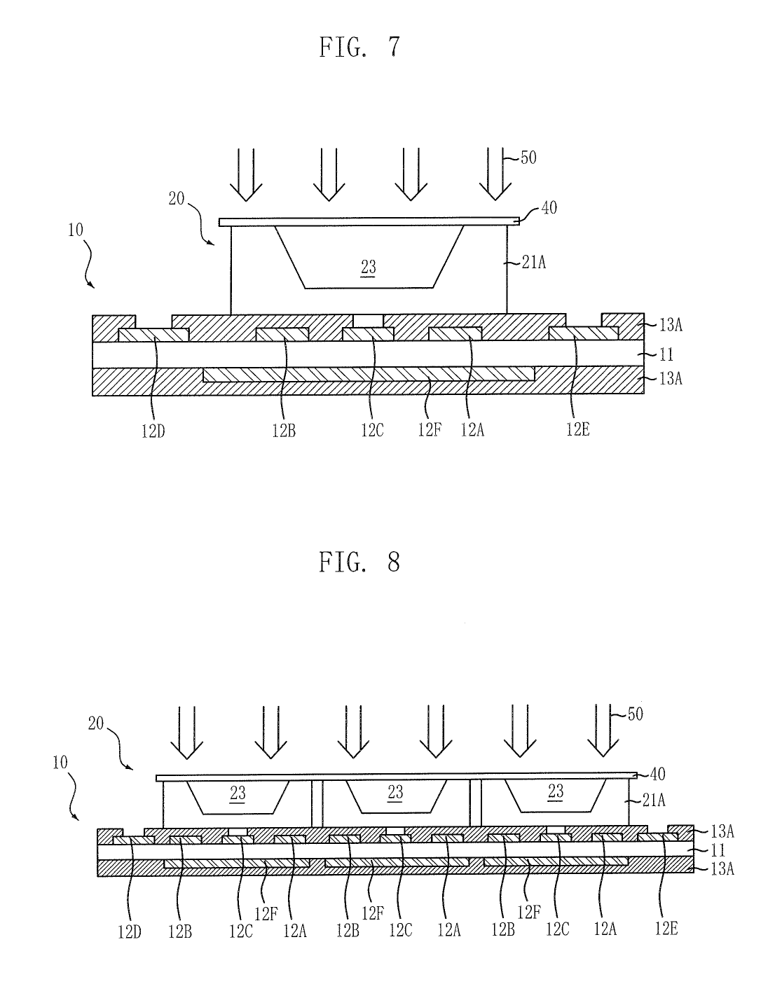

FIG. 7 is a cross-sectional view illustrating a DNA amplification chip and a method for manufacturing the same in accordance with a fourth embodiment of the present invention.

Referring to FIG. 7, the DNA amplification chip differs from the DNA amplification chip of FIG. 5 in that a flat cover 40 is used instead of the inorganic oil cover 30. Since other structures except the flat cover 40 are similar to those of the DNA amplification chip illustrated in FIG. 5, their detailed description will be omitted for conciseness.

Referring to FIG. 7, the DNA amplification chip uses a flat cover 40 for covering the silicon micro-chamber 20. By applying a pressure 50 to the flat cover 40 during the DNA amplification process using PCR, expansion of bubbles generated during the heating within the reaction chamber 23 can be suppressed and the evaporation of genetic sample can be prevented.

FIG. 8 is a cross-sectional view illustrating a DNA amplification chip array in which a plurality of DNA amplification chips of FIG. 7 are arranged. As illustrated in FIG. 8, the DNA amplification chip array can be manufactured in a batch manner by using the method for manufacturing the single DNA amplification chip illustrated in FIG. 7.

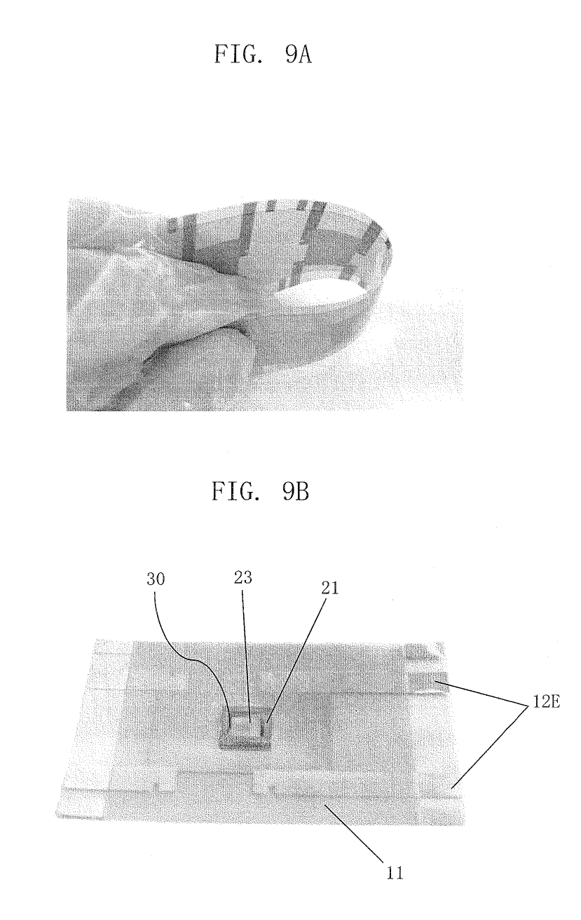

FIG. 9A is a photograph of the microfabricated thermal device illustrated in FIG. 1. Specifically, a plastic-based microfabricated thermal device is formed on a polyimide plastic film by using an FPC process. FIG. 9B is a photograph of the DNA amplification chip illustrated in FIG. 5.

The microfabricated thermal device of FIG. 9A is manufactured using a transparent polyimide plastic substrate having a thickness of 70 μm. Although not shown, a heater, an electrode, and a temperature sensor are formed on the top surface of the plastic substrate, and a variety of devices such as a heat diffusion layer are formed in fine pattern type on the bottom surface of the plastic substrate. The DNA amplification chip of FIG. 9B is manufactured by attaching the silicon micro-chamber to the microfabricated thermal device of FIG. 9A. The pad 12E formed on the plastic substrate 11, and the silicon substrate 21A and the reaction chamber 23 of the silicon micro-chamber are illustrated in FIG. 9B. In addition, the inorganic oil cover 30 is coupled to the reaction chamber 23.

Characteristics of the DNA amplification chip manufactured by the third embodiment of FIG. 5 will be described below.

A typical PCR method was used for comparing amplification characteristics of the DNA amplification chip illustrated in FIG. 5.

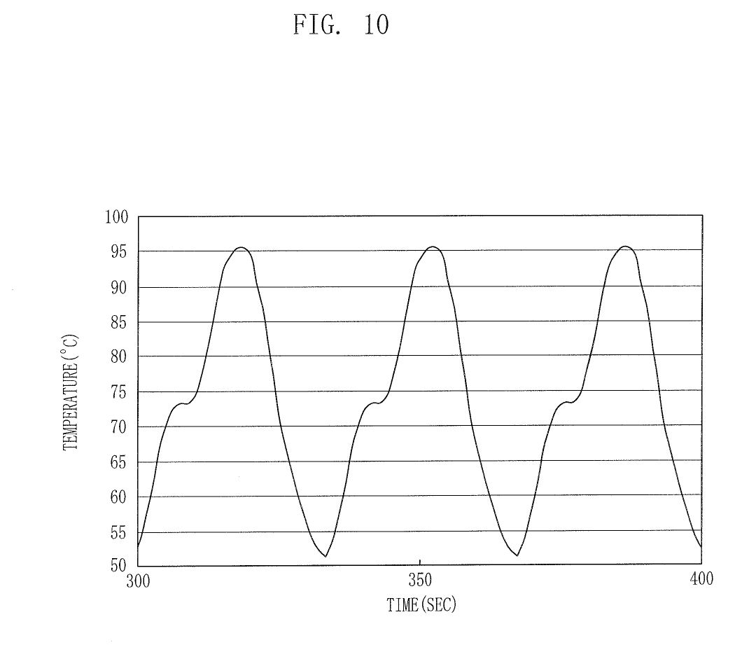

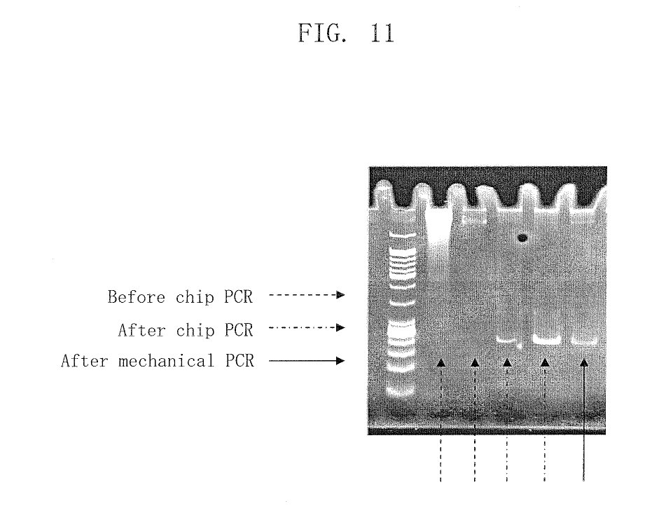

FIG. 10 is a graph illustrating a temperature-time response characteristic of a typical PCR method. FIG. 11 is a photograph illustrating comparative analysis of PCR results, which are obtained using a fluorescent photography obtained through an electrophoresis, before and after a temperature control of PCR is performed on the DNA amplification chip of FIG. 5, and after the temperature control is performed in a mechanical PCR apparatus. The first case is referred to as “before a chip PCR”, the second case is referred to as “after a chip PCR”, and the third case is referred to as “after a mechanical PCR”.

A breast cancer suppressor gene “BRCA1” was used as the sample for the PCR amplification used in each experimental group before the chip PCR and the mechanical PCR. Each PCR procedure was equally applied to each experimental group. That is, after blood sampling, BRCA1 was extracted from the sampled blood and a genomic DNA gene amplification was carried out. The amplification procedure was carried out by denaturizing a DNA strand at 95° C., annealing the DNA strand at 54° C., and extending DNA synthesis at 72° C. for about 18 minutes during 30 cycles.

As illustrated in FIG. 11, the DNA amplification result of the DNA amplification chip of FIG. 5 is very similar to or clearer than the result obtained by a general mechanical PCR. That is, it can be seen from the fluorescent photograph obtained through the electrophoresis that the DNA amplification chip in accordance with the third embodiment of the present invention exhibits excellent DNA amplification characteristics.

The present invention can obtain the following effects.

First, a manufacturing cost can be significantly reduced by manufacturing a microfabricated thermal device using a thin plastic substrate, which is cheaper than silicon or glass. Further, since a heating zone is defined in a portion of the plastic substrate, temperature can be uniformly controlled with low power, and a variety of specimens can be rapidly thermally treated, reacted and analyzed.

Second, thermal mass can be reduced by manufacturing a microfabricated thermal device using an insulating plastic substrate, which has a small thermal mass and a thickness ranging from approximately 1 μm to approximately 500 μm.

Third, fine patterns are formed on the plastic substrate by using the semiconductor fabrication technology such as a photolithography process, and fine devices such as a heater, a temperature sensor, an electrode, and pads, are manufactured using the fine patterns. Therefore, the device can be manufactured using the general semiconductor manufacturing apparatus, without developing new fabrication technology. Consequently, the manufacturing process is simplified and the manufacturing development cost can be saved.

Fourth, thermal uniformity can be enhanced by forming a thermal diffusion layer on the bottom surface of the plastic substrate where the fine devices such as the heater, the temperature sensor, the electrode, and the pad are formed.

Fifth, thermal uniformity and response time can be enhanced by manufacturing the silicon micro-chamber with the reaction chamber by using silicon matching with thermal characteristic of the plastic-based microfabricated thermal device.

Sixth, since the DNA amplification chip is manufactured by attaching the microfabricated thermal device and the silicon micro-chamber, it can be applied to a variety of bio-devices requiring fine and accurate temperature control, e.g., PCR chips, protein chips, drug delivery systems, DNA micro-devices, micro biological/chemical reactors, etc.

While the present invention has been described with respect to the specific embodiments, it will be apparent to those skilled in the art that various changes and modifications may be made without departing from the spirit and scope of the invention as defined in the following claims.

What is claimed is:

1. A plastic-based microfabricated thermal device, comprising:
a plastic substrate;
a heating unit disposed on the top surface of the plastic substrate to supply heat to the plastic substrate;
a sensing unit disposed on the top surface of the plastic substrate to detect heat; and
a diffusing unit disposed on the bottom surface of the plastic substrate to diffuse heat to the plastic substrate.

2. The plastic-based microfabricated thermal device of claim 1, further comprising insulating layers disposed on the top and bottom surfaces of the plastic substrate to cover the heating unit, the sensing unit, and the diffusing unit.

3. The plastic-based microfabricated thermal device of claim 1, wherein the heating unit includes:
a heater disposed on the top surface of the plastic substrate;
an electrode disposed on the top surface of the plastic substrate and connected to the heater; and
a pad disposed on the top surface of the plastic substrate to supply a power to the heater through the electrode.

4. The plastic-based microfabricated thermal device of claim 1, wherein the diffusing unit is formed of the same material as the heating unit and the sensing unit.

5. The plastic-based microfabricated thermal device of claim 1, wherein the heating unit, the sensing unit, and the diffusing unit are formed of metal patterns.

6. The plastic-based microfabricated thermal device of claim 1, wherein the diffusing unit is formed of metal or graphite.

7. The plastic-based microfabricated thermal device of claim 1, wherein the plastic substrate is formed of a polymer or a mixture containing the polymer.

8. The plastic-based microfabricated thermal device of claim 1, wherein the plastic substrate is formed of one material selected from the group consisting of Cyclo Olefin Copolymer (COC), PolyMethylMethAcrylate (PMMA), PolyCarbonate (PC), Cyclo Olefin Polymer (COP), Liquid Crystalline Polymers (LCP), PolyDiMethylSiloxane (PDMS), PolyAmide (PA), PolyEthylene (PE), PolyImide (PI), PolyPropylene (PP), PolyPhenylene Ether (PPE), PolyStyrene (PS), PolyOxyMethylene (POM), PolyEtherEtherKetone (PEEK), PolyEthylenephThalate (PET), PolyTetraFluoroEthylene (PTFE), PolyVinylChloride (PVC), PolyVinyliDeneFluoride (PVDF), PolyButyleneTerephtalate (PBT), Fluorinated EthyleneproPylene (FEP), and PerFluorAlkoxyalkane (PFA), and mixtures thereof.

9. The plastic-based microfabricated thermal device of claim 1, wherein the plastic substrate is coated with a liquid inorganic or organic thin film.

10. The plastic-based microfabricated thermal device of claim 1, wherein the plastic substrate includes a concave region in which the heating unit and the sensing unit are formed.

11. The plastic-based microfabricated thermal device of claim 1, wherein the plastic substrate has a thickness ranging from approximately 1 μm to approximately 500 μm.

12. The plastic-based microfabricated thermal device of claim 1, wherein the plastic substrate has a surface roughness ranging from approximately 0.1 nm to approximately 500 nm.

13. A DNA amplification chip, comprising:
a plastic-based microfabricated thermal device including:
a plastic substrate;
a heating unit disposed on the top surface of the plastic substrate to supply heat to the plastic substrate;
a sensing unit disposed on the top surface of the plastic substrate to detect heat; and
a diffusing unit disposed on the bottom surface of the plastic substrate to diffuse heat to the plastic substrate;
a silicon micro-chamber including a concave region and attached to the microfabricated thermal device, with the concave region being directed upwards; and
a cover disposed to cover the concave region of the silicon micro-chamber, thereby defining a reaction chamber.

14. The DNA amplification chip of claim 13, wherein the silicon micro-chamber is adhered to an insulating layer of the microfabricated thermal device by an adhesive material.

15. The DNA amplification chip of claim 13, wherein the cover is formed of inorganic oil or flat plate.

16. The DNA amplification chip of claim 13, wherein the microfabricated thermal devices, the silicon micro-chamber, and the cover are provided in plurality and are arrayed on a single plastic substrate.

17. A method for manufacturing a plastic-based microfabricated thermal device, comprising the steps of:
a) preparing a plastic substrate;
b) forming a heater, an electrode, a pad, and a temperature sensor on the top surface of the plastic substrate;
c) forming a heat diffusion layer on the bottom surface of the plastic substrate;
d) forming insulating layers on the top and bottom surfaces of the plastic substrate to cover the heater, the electrode, the pad, the temperature sensor, and the heat diffusion layer; and
e) etching the insulating layers to expose predetermined portions of the electrode and the pad.

18. The method of claim 17, wherein the step b) includes the steps of:
b1) depositing a metal layer on the plastic substrate; and
etching the metal layer to form a metal pattern.

19. The method of claim 17, wherein the step c) includes the steps of:
c1) forming a metal layer on the bottom surface of the plastic substrate; and
c2) etching the metal layer to form a metal pattern.

20. The method of claim 17, wherein the plastic substrate is formed of a polymer or a mixture containing the polymer.

21. The method of claim 17, wherein the plastic substrate is formed of one material selected from the group consisting of Cyclo Olefin Copolymer (COC), PolyMethylMethAcrylate (PMMA), PolyCarbonate (PC), Cyclo Olefin Polymer (COP), Liquid Crystalline Polymers (LCP), PolyDiMethylSiloxane (PDMS), PolyAmide (PA), PolyEthylene (PE), PolyImide (PI), PolyPropylene (PP), PolyPhenylene Ether (PPE), PolyStyrene (PS), PolyOxyMethylene (POM), PolyEtherEtherKetone (PEEK), PolyEthylenephThalate (PET), PolyTetraFluoroEthylene (PTFE), PolyVinylChloride (PVC), PolyVinyliDeneFluoride (PVDF), PolyButyleneTerephtalate (PBT), Fluorinated EthyleneproPylene (FEP), and PerFluorAlkoxyalkane (PFA), and mixtures thereof.

22. The method of claim 17, wherein the plastic substrate is coated with a liquid inorganic or organic thin film.

23. The method of claim 17, wherein the plastic substrate is formed by an injection molding, an extrusion molding, a hot embossing, a stereolithography, a laser ablation, a rapid prototyping, a founding, a silk screen, or a machining.

24. A method for manufacturing a DNA amplification chip, comprising the steps of:
a) providing a plastic-based microfabricated thermal device, the plastic-based microfabricated thermal device being formed by preparing a plastic substrate, forming a heater, an electrode, a pad, and a temperature sensor on the top surface of the plastic substrate, forming a heat diffusion layer on the bottom surface of the plastic substrate, forming insulating layers on the top and bottom surfaces of the plastic substrate to cover the heater, the electrode, the pad, the temperature sensor, and the heat diffusion layer, and etching the insulating layers to expose predetermined portions of the electrode and the pad;
b) forming a silicon micro-chamber having a concave region and attaching the silicon micro-chamber to the top surface of the microfabricated thermal device; and
c) covering the concave region by a cover to form a reaction chamber.

25. The method of claim 25, wherein the cover is formed of inorganic oil or flat plate.

26. The method of claim 24, wherein the step b) includes the steps of:
b1) forming an insulating layer on a silicon substrate;
b2) etching the insulating layer to form an etch mask; and
forming the concave region by etching the silicon substrate to a predetermined depth by an etching process using the etch mask.

27. The method of claim 24, wherein the silicon micro-chamber is attached to the microfabricated thermal device by an adhesive material.
